(19) United States
(12) Patent Application Publication
(10) Pub. No.: US 2025/0241680 A1
(43) Pub. Date: Jul. 31, 2025

(54) BLOOD VESSEL PUNCTURE APPARATUS AND METHOD FOR CONTROLLING BLOOD VESSEL PUNCTURE SYSTEM

(71) Applicant: TERUMO KABUSHIKI KAISHA, Tokyo (JP)

(72) Inventors: Kazuki NAKASONE, Fujinomiya-shi (JP); Takito INUKAI, Isehara-shi (JP); Takumi FUKUDA, Hadano-shi (JP); Yoichiro KUWANO, Princeton, NJ (US)

(73) Assignee: TERUMO KABUSHIKI KAISHA, Tokyo (JP)

(21) Appl. No.: 19/089,291

(22) Filed: Mar. 25, 2025

Related U.S. Application Data
(63) Continuation of application No. PCT/JP2023/034444, filed on Sep. 22, 2023.

(30) Foreign Application Priority Data
Sep. 28, 2022 (JP) 2022-154398

(51) Int. Cl. A61B 17/34; A61B 17/00; A61B 90/00

(52) U.S. Cl. CPC A61B 17/3494; A61B 17/3403; A61B 2017/00057; A61B 2017/00075; A61B 2017/00694; A61B 2017/00778; A61B 2090/064

(57) ABSTRACT

A blood vessel puncture apparatus capable of detecting vasospasm and a method for controlling a blood vessel puncture system. The blood vessel puncture apparatus connectable to an imaging unit that measures a blood vessel diameter and a drive unit that moves a needle that performs puncture includes a control unit that receives information of a measurement result from the imaging unit and controls an operation of the drive unit, in which the control unit determines whether a blood vessel contracts from the measurement result acquired from the imaging unit, and controls the drive unit to start, continue, or stop movement of the needle in a case where it is determined that the blood vessel contracts.

### CROSS-REFERENCES TO RELATED APPLICATIONS

This application is a continuation of International Application No. PCT/JP2023/034444 filed on Sep. 22, 2023, which claims priority to Japanese Application No. 2022-154398 filed on Sep. 28, 2022, the entire content of both of which is incorporated herein by reference.

### TECHNOLOGICAL FIELD

The present disclosure generally relates to a blood vessel puncture apparatus capable of automatically puncturing a blood vessel and a method for controlling a blood vessel puncture system.

### BACKGROUND DISCUSSION

In order to secure an access route to a blood vessel for drug administration and endovascular treatment, blood vessel puncture is performed in which a human body is punctured with a needle having a sharp needle tip, the needle being covered by a flexible outer tube. The access route can be secured using the outer tube, since only the needle is removed after the needle and the outer tube reach the inside of the blood vessel. In the blood vessel puncture, an operator cannot visually observe the blood vessel from a skin surface, and thus, a position of the blood vessel is estimated by standard knowledge of blood vessel running and skill such as tactile perception of blood vessel pulsation.

In recent years, there is a device that performs blood vessel puncture automatically (see, for example, U.S. Pat. No. 9,364,171).

Meanwhile, when vasospasm (spasm) occurs during blood vessel puncture, it is difficult to secure a lumen in a blood vessel for inserting a device such as a sheath to be inserted into the blood vessel. Therefore, when vasospasm (i.e., narrowing of the blood vessel) occurs, there is a case where puncture cannot be performed, or even if puncture can be performed, it may be difficult to insert the device into the blood vessel after puncture is performed.

In a case where vasospasm occurs, vasodilator administration may be performed. Therefore, in a case where vasospasm occurs, it is desirable to be able to detect the vasospasm.

### SUMMARY

A blood vessel puncture apparatus is disclosed that is capable of detecting vasospasm and a method for controlling a blood vessel puncture system.
(1) A blood vessel puncture apparatus is disclosed, which is connectable to a measurement unit for measuring a blood vessel diameter and a drive unit for moving a needle for puncturing, the blood vessel puncture apparatus including: a control unit that receives information of a measurement result from the measurement unit and controls an operation of the drive unit. The control unit determines whether the blood vessel contracts from a measurement result acquired from the measurement unit, and controls the drive unit to start, continue, or stop movement of the needle in a case where it is determined that the blood vessel contracts.
(2) A blood vessel puncture apparatus is disclosed, which is connectable to a measurement unit for measuring a blood vessel diameter, a drive unit for moving a needle for puncturing, and an information transmission unit for transmitting information, the blood vessel puncture apparatus including: a control unit that receives information of a measurement result from the measurement unit and controls an operation of the drive unit. The control unit determines whether a blood vessel contracts from a measurement result acquired from the measurement unit, and causes the information transmission unit to transmit information indicating a warning in a case where it is determined that the blood vessel contracts.

The blood vessel puncture apparatus according to (1) detects vasospasm by detecting contraction of a blood vessel by the measurement unit, and can start, continue, or stop puncture.

The blood vessel puncture apparatus according to (2) can detect vasospasm by detecting contraction of a blood vessel by the measurement unit and transmit a warning.
(3) In the blood vessel puncture apparatus according to (1) or (2), the measurement unit may be an imaging unit that comes into contact with a skin surface to acquire a cross-sectional image of a human body, and the control unit may calculate a blood vessel diameter from a cross-sectional image acquired from the imaging unit, and determine, in a case where the blood vessel diameter is equal to or less than a threshold, that the blood vessel contracts and vasospasm occurs. As a result, the blood vessel puncture apparatus can effectively detect the vasospasm from the cross-sectional image acquired from the imaging unit.
(4) In the blood vessel puncture apparatus according to (1) or (2), the measurement unit may be a force sensor that detects a force acting on the needle, and the control unit may specify a first peak when the needle punctures a front wall of a blood vessel and a second peak when the needle punctures a back wall of the blood vessel after the first peak from a detection result of the force sensor, calculate an inter-wall distance that is a movement amount of the needle between the first peak and the second peak, and determine, in a case where the inter-wall distance is equal to or less than a threshold, that the blood vessel contracts and vasospasm occurs. As a result, the blood vessel puncture apparatus can effectively detect the vasospasm from the detection result of the force acquired from the force sensor.
(5) In the blood vessel puncture apparatus according to (3), the control unit may calculate the threshold by multiplying a blood vessel diameter specified from a cross-sectional image acquired from the imaging unit by a predetermined ratio before starting a puncture operation by the drive unit. As a result, since the blood vessel puncture apparatus calculates the threshold from the actual blood vessel diameter before puncture, the vasospasm can be effectively detected by appropriately setting the ratio.
(6) In the blood vessel puncture apparatus according to (4), before starting a puncture operation by the drive unit, the control unit may calculate the threshold by multiplying a distance between a front wall and a back wall of a blood vessel specified from a cross-sectional image acquired from an imaging unit that comes into contact with a skin surface and acquires a cross-sectional image of a human body by a predetermined ratio. Accordingly, since the blood vessel puncture apparatus calculates the threshold from the distance between the front wall and the back wall of the actual blood vessel before puncture, the vasospasm can be effectively detected by appropriately setting the ratio.
(7) In the blood vessel puncture apparatus according to any one of (1) to (6), the control unit may determine whether the blood vessel contracts before starting a puncture operation by the drive unit or during the puncture operation. As a result, the blood vessel puncture apparatus can quickly detect the vasospasm even before or during the puncture of the blood vessel, and can safely cope with the vasospasm after the detection with time.
(8) A method is disclosed for controlling a blood vessel puncture system, the blood vessel puncture system including a measurement unit that measures a blood vessel diameter, a drive unit that moves a needle for puncturing, and a blood vessel puncture apparatus including a control unit that receives information of a measurement result from the measurement unit and controls an operation of the drive unit, the method including: a step of determining whether a blood vessel contracts from a measurement result acquired from the measurement unit; and a step of controlling the drive unit to start, continue, or stop movement of the needle in a case where it is determined that the blood vessel contracts.

The method for controlling the blood vessel puncture system according to (8) can detect vasospasm by detecting contraction of the blood vessel by the measurement unit and start, continue, or stop puncture.
(9) A method is disclosed for controlling a blood vessel puncture system, the blood vessel puncture system including: a measurement unit that measures a blood vessel diameter; a drive unit that moves a needle for puncturing; an information transmission unit that transmits information; and a blood vessel puncture apparatus including a control unit that receives information on a measurement result from the measurement unit and controls an operation of the drive unit, the method including: a step of determining whether a blood vessel has contracted from the measurement result acquired from the measurement unit; and a step of causing the information transmission unit to transmit information indicating a warning when it is determined that the blood vessel has contracted.

In the method for controlling the blood vessel puncture system according to (9), vasospasm can be detected by detecting contraction of the blood vessel by the measurement unit and a warning can be transmitted.

### BRIEF DESCRIPTION OF THE DRAWINGS

FIG. 1 is a side view of a blood vessel puncture system including a blood vessel puncture apparatus according to a first embodiment.

FIG. 2 is a top view of the blood vessel puncture system, which illustrates a positional relationship with an arm whose cross-sectional image is to be acquired.

FIG. 3 is a configuration diagram of the blood vessel puncture system.

FIG. 4 is a schematic view illustrating an example of an image acquired by an imaging unit.

FIG. 5 is a side view illustrating the blood vessel puncture system immediately before puncture in a state where a probe body is inclined with respect to a skin surface.

FIG. 6 is a top view illustrating the blood vessel puncture system immediately before puncture in a state where the probe body is inclined with respect to the skin surface.

FIG. 7 is a schematic view for explaining a positional relationship between a blood vessel and a puncture portion in a state where a needle punctures a front wall.

FIG. 8 is a schematic view for explaining a positional relationship between a blood vessel and a puncture portion in a state where a needle punctures a back wall.

FIG. 9 is a flowchart illustrating a flow of control in a control unit according to the first embodiment.

FIG. 10 is a flowchart illustrating a flow of control in a control unit according to a second embodiment.

FIG. 11 is a graph illustrating an example of a relationship between displacement of the needle in the puncture direction at the time of puncture and a force acting on the needle.

### DETAILED DESCRIPTION

Set forth below with reference to the accompanying drawings is a detailed description of embodiments of a blood vessel puncture apparatus capable of automatically puncturing a blood vessel and a method for controlling a blood vessel puncture system. Note that, dimensional ratios in the drawings are sometimes exaggerated and different from actual ratios for convenience of description.

### First Embodiment

A blood vessel puncture apparatus 11 according to the first embodiment of the present invention is an apparatus that constitutes a part of a blood vessel puncture system 10 and controls the operation of the blood vessel puncture system 10. The blood vessel puncture system 10 is used when puncturing an arm H of a human body to acquire a cross-sectional image of the arm H, detect a position of an artery to be punctured, and automatically puncture the artery.

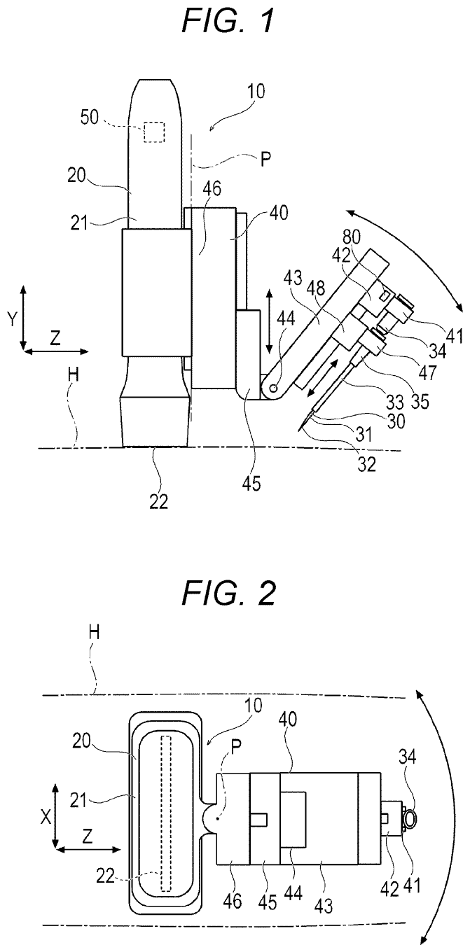

As illustrated in FIGS. 1 to 3, the blood vessel puncture system 10 includes: a probe body 20 having an imaging unit 22 (measurement unit) that comes into contact with a skin surface and acquires a cross-sectional image of a human body; a puncture unit 30 that performs puncture; a drive unit 40 that moves the puncture unit 30 with respect to the probe body 20; an inclination detection unit 50 that detects an inclination angle of the probe body 20; a display unit 70 capable of displaying the cross-sectional image; a force sensor 80 that detects a force acting on the puncture unit 30; and a blood vessel puncture apparatus 11. The blood vessel puncture apparatus 11 includes a control unit 60 that performs image analysis of the cross-sectional image, controls the drive unit 40, and the like.

The probe body 20 includes a vertically elongated handle portion 21 gripped by an operator, an imaging unit 22 disposed at a lower end of the handle portion 21, a transmitter 23 that transmits a signal from the control unit 60 to the imaging unit 22, and a receiver 24 that transmits a signal from the imaging unit 22 to the control unit 60.

The imaging unit 22 is provided so as to extend over substantially the entire width at the central portion of a lower surface of the probe body 20. The imaging unit 22 is an echographic apparatus that includes a transducer that generates an ultrasound wave and obtain the cross-sectional image of the inside of the human body by detecting a reflected wave of the ultrasound wave. In the present embodiment, the cross-sectional image orthogonal to an axial direction of the blood vessel is acquired, and thus, the imaging unit 22 is disposed such that a length direction is orthogonal to a length direction of the arm H.

The transmitter 23 transmits a signal from the control unit 60 to the imaging unit 22 in order to output an ultrasound wave from the imaging unit 22. The receiver 24 transmits, to the control unit 60, a signal output from the imaging unit 22 receiving the reflected wave.

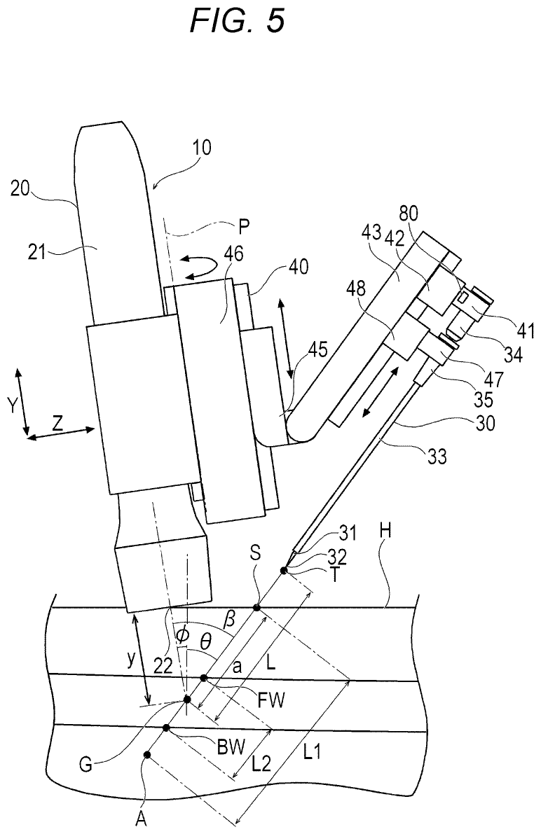

The inclination detection unit 50 is connected to the control unit 60. The inclination detection unit 50 can be, for example, a gyro sensor, and can detect an inclination of the probe body 20. A reference of the inclination is a perpendicular direction orthogonal to the horizontal direction. Since an upper surface of the arm H when performing the puncture is set along the horizontal direction, an inclination of the blood vessel puncture system 10 with respect to a perpendicular line of the skin surface can be detected by detecting the inclination with respect to the perpendicular direction by the inclination detection unit 50. In the present example, it is assumed that the inclination detection unit 50 detects that the blood vessel puncture system 10 is inclined at an angle of p as illustrated in FIG. 5. Note that the inclination detection unit 50 is not limited to the gyro sensor, and may be, for example, a camera that captures an image of the skin surface of the arm H. In this case, the control unit 60 can detect the inclination φ of the probe body 20 from a result of the image capturing by the inclination detection unit 50 using a technique such as machine learning or deep learning. In addition, the inclination detection unit 50 is not necessarily provided in the probe body 20 of the blood vessel puncture system 10.

As illustrated in FIGS. 1 and 5, the puncture unit 30 includes a needle 31 made of metal and having a sharp needle tip 32 formed at a distal end, and a flexible tubular outer tube 33 disposed so as to cover an outer peripheral surface of the needle 31. The needle 31 may be either solid or hollow.

The needle tip 32 is a portion having a blade surface inclined with respect to an axial center on a side closer to the distal end than a portion where the outer diameter of the needle 31 is constant. Alternatively, the needle tip 32 may be a portion whose outer diameter decreases toward the most distal end that is sharp.

As illustrated in FIGS. 1 and 5, the needle tip 32 protrudes from the outer tube 33 in a state where the outer tube 33 covers the outer side of the needle 31. A needle hub 34 is fixed to a proximal end of the needle 31. A tubular outer tube hub 35 is fixed to a proximal end of the outer tube 33.

As illustrated in FIGS. 1 and 2, the drive unit 40 includes: a first holding portion 41 that holds the needle hub 34; a first linear movement portion 42 that linearly moves the first holding portion 41; a second holding portion 47 that holds the outer tube hub 35; a second linear movement portion 48 that linearly moves the second holding portion 47; an inclination portion 43 that inclines the first holding portion 41 and the second holding portion 47; a third linear movement portion 45 that moves the inclination portion 43 in a length direction of the probe body 20; and a rotation portion 46 that rotates the third linear movement portion 45 about a predetermined rotation axis P.

The first holding portion 41 can detachably hold the needle hub 34. The first holding portion 41 can be, for example, a clamp that can perform holding so as to sandwich the needle hub 34.

The first linear movement portion 42 can linearly move the first holding portion 41 holding the needle hub 34 of the puncture unit 30 forward and backward along an extending direction (puncture direction) of the needle 31. The first linear movement portion 42 is used to adjust a position of the needle 31 and puncture the blood vessel with the needle 31. The first linear movement portion 42 can include, for example, a rotational drive source such as a motor whose driving can be controlled by the control unit 60, and a structure (for example, a feed screw mechanism) that converts a rotational motion of the rotational drive source into a linear motion.

The second holding portion 47 can detachably hold the outer tube hub 35. The second holding portion 47 can be, for example, a clamp that can perform holding so as to sandwich the outer tube hub 35.

The second linear movement portion 48 can linearly move the second holding portion 47 holding the outer tube hub 35 of the puncture unit 30 forward and backward along an extending direction (puncture direction) of the outer tube 33. The second linear movement portion 48 can adjust a position of the outer tube and push the outer tube 33 into the puncture hole formed by the needle 31. The second linear movement portion 48 can include, for example, a rotational drive source such as a motor whose driving can be controlled by the control unit 60, and a structure (for example, a feed screw mechanism) that converts a rotational motion of the rotational drive source into a linear motion.

The inclination portion 43 can incline the first linear movement portion 42 and the second linear movement portion 48. The inclination portion 43 is used to change puncture angles of the needle 31 and the outer tube 33 with respect to a surface of a skin of the patient. The inclination portion 43 includes a hinge 44 whose angle can be changed, and a rotational drive source such as a motor whose driving can be controlled by the control unit 60 in order to change the angle of the hinge 44.

The third linear movement portion 45 is used to bring the puncture unit 30 close to or away from the skin of the patient. The third linear movement portion 45 can linearly move the inclination portion 43 forward and backward along an extending direction of the probe body 20. The third linear movement portion 45 can include, for example, a rotational drive source such as a motor whose driving can be controlled by the control unit 60, and a structure (for example, a feed screw mechanism) that converts a rotational motion of the rotational drive source into a linear motion.

The rotation portion 46 is used to change a direction of the needle 31 when the third linear movement portion 45 is viewed substantially perpendicularly to the surface of the skin of the patient. The rotation portion 46 can rotate the inclination portion 43 about the rotation axis P parallel to the length direction of the probe body 20. The rotation portion 46 can include, for example, a rotational drive source such as a motor whose driving can be controlled by the control unit 60.

The drive sources used for the first linear movement portion 42, the second linear movement portion 48, the third linear movement portion 45, and the rotation portion 46 are preferably configured to be able to control rotation and displacement with relatively high accuracy while being grasped by the control unit 60, and are, for example, servomotors.

The force sensor 80 detects a force F in the puncture direction acting on the needle 31. The force sensor 80 can be disposed, for example, in the first holding portion 41, but a place where the force sensor is disposed is not limited as long as the force F can be detected. The force sensor 80 transmits a detected signal to the control unit 60.

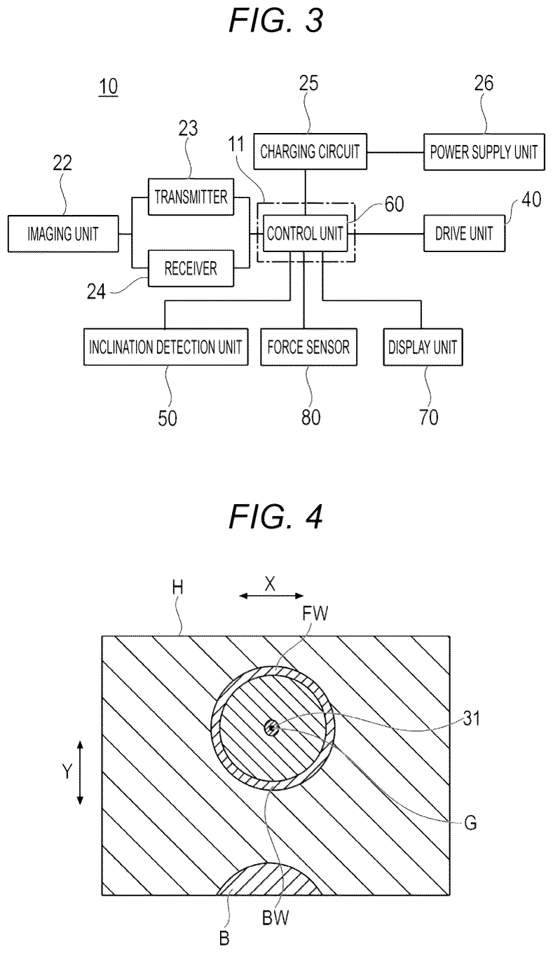

As illustrated in FIGS. 1 and 3, the control unit 60 transmits a signal to the imaging unit 22 via the transmitter 23 and causes the imaging unit 22 to output an ultrasound wave. In addition, the control unit 60 can form a cross-sectional image from a signal obtained from the imaging unit 22 via the receiver 24. Further, the control unit 60 can cause the display unit 70 to display the obtained cross-sectional image. Furthermore, the control unit 60 can perform arithmetic processing such as image analysis from the information of the cross-sectional image to specify the position of the blood vessel in the image. Furthermore, the control unit 60 receives a detection signal (detection result) indicating the force F received by the needle 31 from the force sensor 80. Further, the control unit 60 can control the operation of the drive unit 40. The control unit 60 includes, as physical configurations, a storage circuit and an arithmetic circuit. The storage circuit can store programs and various parameters. The arithmetic circuit can perform arithmetic processing.

The control unit 60 is connected to a power supply unit 26 including a rechargeable battery via a charging circuit 25. In addition, the control unit 60 is connected to the inclination detection unit 50. The control unit 60 may be disposed in the probe body 20 or the drive unit 40, or may be configured separately from the probe body 20 or the drive unit 40.

The control unit 60 acquires a cross-sectional image as illustrated in FIG. 4 from the imaging unit 22. It is assumed that a lateral direction in the cross-sectional image, that is, a width direction of the arm H is an X direction, a longitudinal direction in the cross-sectional image, that is, a depth direction of the arm H is a Y direction, and a direction orthogonal to the paper surface of the cross-sectional image, that is, the length direction of the arm H is a Z direction. Coordinates of an upper left point in the cross-sectional image are set as a start point (0, 0, 0). In the cross-sectional image, the wall of the blood vessel on the side close to the skin to be punctured is a front wall FW, and the wall of the blood vessel on the side away from the skin to be punctured is a back wall BW. A radius B may be disposed behind the back wall BW. The puncture by the needle 31 is performed in a double wall puncture (DWP) so as to pass through the front wall FW, a barycenter G, and the back wall BW of the blood vessel. However, since the needle 31 punctures obliquely with respect to the extending direction of the blood vessel, in a case where the range including the barycenter G of the blood vessel is observed from the cross-sectional image, the needle tip 32 puncturing the back wall BW cannot be observed from the cross-sectional image.

As illustrated in FIGS. 3 and 4, the display unit 70 is a monitor or the like capable of displaying the cross-sectional image.

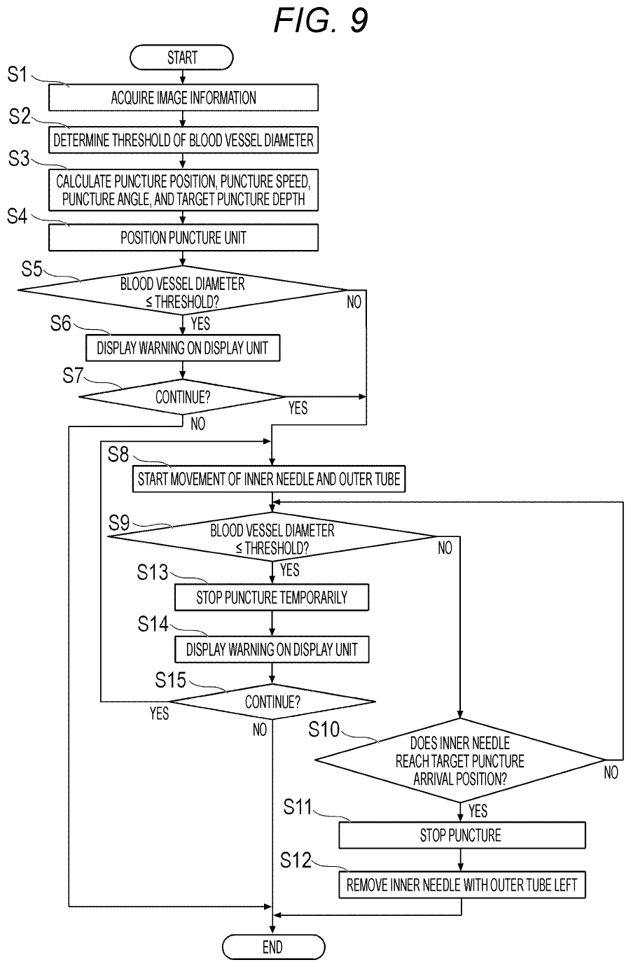

Next, a method for puncturing a blood vessel using the blood vessel puncture system 10 will be described with reference to a flowchart of the control unit 60 illustrated in FIG. 9. As illustrated in FIGS. 1 and 2, the blood vessel puncture system 10 is used in contact with the skin surface.

The control unit 60 receives an instruction to start automatic puncture from the operator by an input means such as a switch, a keyboard, or a mouse connected to the control unit 60. The control unit 60 acquires image information from the imaging unit 22 via the receiver 24 (step S1). The control unit 60 forms a cross-sectional image from the image information. The control unit 60 performs image analysis on the obtained cross-sectional image to specify the position of the blood vessel in the image, and specifies the inner diameter of the blood vessel to determine a threshold (step S2).

The threshold is a value used to determine whether vasospasm has occurred. The threshold is a value obtained by multiplying the inner diameter of the blood vessel specified from the cross-sectional image by a ratio. Although the artery repeats expansion and contraction, the inner diameter of the blood vessel specified from the cross-sectional image is preferably, but not limited to, the inner diameter at the time of contraction. Since puncturing is performed to create a passageway through which the device is inserted into the blood vessel, it is desirable to assess the inner diameter of the blood vessel at the time of contraction when the passageway is the narrowest. The ratio can be set in the control unit 60 in advance before the procedure is started. The ratio is more than 0% and less than 100%, for example, 70%. The inner diameter of the blood vessel specified from the cross-sectional image may be subjected to arithmetic processing of averaging measurement results at a plurality of points, for example. The threshold may not be calculated from the inner diameter of the blood vessel specified from the cross-sectional image. For example, the threshold may be calculated from the outer diameter of the blood vessel specified from the cross-sectional image, or may be calculated from a value between the outer diameter and the inner diameter of the blood vessel specified from the cross-sectional image. Alternatively, the threshold may be a value obtained by multiplying the outer diameter of the device to be inserted into the blood vessel after puncture by a preset ratio. Furthermore, the threshold may be a specific value determined by the operator.

Furthermore, the control unit 60 causes the display unit 70 to display the cross-sectional image. The position of the blood vessel to be specified includes the position of the front wall FW, the position of the back wall BW, and the position of the barycenter G of the blood vessel. In order to specify the position of the blood vessel, the inner diameter of the blood vessel, and the like in the image, the control unit 60 can prepare a large number of images of the same type and use a technique such as machine learning or deep learning. In addition, it is also possible to detect a region with blood flow by the Doppler method in the imaging unit 22 and recognize the region as a region of the blood vessel.

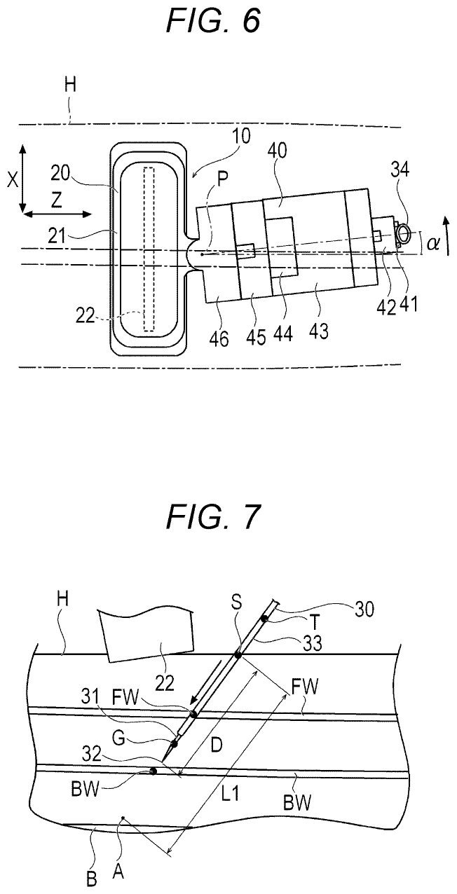

Next, as illustrated in FIGS. 5 and 6, the control unit 60 calculates a puncture position S, a puncture speed, a puncture angle θ, and a target puncture depth L1 of the skin surface from the position information of the blood vessel (step S3). The puncture angle θ is an angle at which the needle 31 at the time of puncture is inclined with respect to a perpendicular line of the skin surface. The puncture angle θ can also be, for example, a preset angle (for example, 30 degrees). The target puncture depth L1 is a distance from the puncture position S on the skin surface to a predetermined position (target puncture arrival position A) after passing through the front wall FW of the blood vessel, the barycenter G of the blood vessel, and the back wall BW of the blood vessel. The target puncture arrival position A is the deepest position where the needle tip 32 of the needle 31 is to arrive. Note that the target puncture arrival position A may be changed by the control unit 60 calculating in the middle of puncture according to the situation at the time of puncture.

In the present embodiment, puncture is performed by a method in which the needle 31 punctures both the front wall FW and the back wall BW of the blood vessel, is retracted, and is pulled out from the back wall BW, that is, a so-called double wall puncture (DWP). However, the puncture may be performed by a method for puncturing only the front wall FW of the blood vessel with the needle 31, a so-called single wall puncture (SWP).

The control unit 60 sets the coordinates of the barycenter G of the detected blood vessel as (x, y, 0). Next, the control unit 60 calculates a position (coordinates) and a posture (angle) of the puncture unit 30 desirable for the puncture. The control unit 60 further calculates a preparation position T and a rotation angle α. The preparation position T is a position of the needle tip 32 immediately before puncture. The rotation angle α is an angle at which the needle 31 at the time of puncture is inclined with respect to the Z direction when the surface of the arm H is viewed from a direction of the perpendicular line. The rotation angle α is set within a range in which the needle tip 32 of the needle 31 can reach the inside of an artery. The preparation position T is set at a certain height from the skin surface. The preparation position T is a position where the needle 31 can reach the inside of the blood vessel on the cross-sectional image by being caused to protrude along the extending direction (puncture direction).

In the cross-sectional image acquired by the control unit 60 from the imaging unit 22, the Y direction is inclined at an angle φ with respect to a perpendicular line of the skin surface. In addition, the control unit 60 acquires an inclination φ of the blood vessel puncture system 10 by the inclination detection unit 50. The control unit 60 sets an upper left end position of the acquired cross-sectional image as a start point (0, 0, 0). With this start point as a reference, the control unit 60 detects the barycenter G of the barycenter of each blood vessel from the cross-sectional image.

For example, coordinates of the barycenter G of the barycenter of one detected blood vessel are set to (x, y, 0), and the rotation angle α is simply set to 0 degrees. A coordinate y1 in the Y direction of the puncture position S on the skin surface can be calculated by y1=y−a·cos(φ+θ) as illustrated in FIG. 5. A coordinate z1 in the Z direction of the puncture position S can be calculated by z1=a·sin(φ+θ). Further, the depth a from the puncture position S to the barycenter G is calculated by a=y·cos φ/cos θ. As a result, the coordinates (x, y1, z1) of the puncture position S and the puncture depth a are defined.

A distance L from the preparation position T where the needle tip 32 is disposed to the barycenter G is set to a value longer than the depth a from the puncture position S to the barycenter G. An angle β between a plane of the cross-sectional image and the puncture direction is obtained by β=θ+φ, and coordinates of the preparation position T can be specified by defining the distance L from the preparation position T to the barycenter G, the rotation angle α, and the angle β. In a case where the coordinates of the preparation position T are (x, y2, z2) and the rotation angle α is simply 0 degrees, the coordinate y2 in the Y direction can be calculated by y2=y−L·cos(φ+θ). The coordinate z2 in the Z direction can be calculated by z2=L·sin(φ+θ).

Next, the control unit 60 controls and drives at least one of the first linear movement portion 42, the second linear movement portion 48, the third linear movement portion 45, the inclination portion 43, or the rotation portion 46 such that the needle 31 satisfies the puncture distance L, the rotation angle α, and the angle β. Therefore, as illustrated in FIGS. 5 and 6, the puncture unit 30 is positioned at the desired position (coordinates) with the desired posture (angle) (step S4). At this time, the needle tip 32 of the needle 31 is disposed at the preparation position T. In order to maintain a relative positional relationship between the needle 31 and the outer tube 33, the first linear movement portion 42 and the second linear movement portion 48 synchronously move in the same direction by the same length.

The control unit 60 acquires image information from the imaging unit 22, forms a cross-sectional image, and specifies the inner diameter of the blood vessel. Next, the control unit 60 determines whether the specified blood vessel inner diameter is equal to or less than a threshold (or less than the threshold) (step S5).

In a case where it is determined that the inner diameter of the blood vessel is equal to or less than the threshold (or less than the threshold) in step S5, the control unit 60 determines that vasospasm may have occurred and displays a warning on the display unit 70 (step S6). Next, the control unit 60 waits for an input to the control unit 60 by the operator who has seen the warning (step S7). The operator checks the situation by viewing the warning, and determines whether to continue the automatic puncture (including continuation for dosing to eliminate vasospasm) or to stop the automatic puncture. Note that the determination as to whether to continue may be performed not by the operator but by the control unit 60. In step S7, in a case where an input for instructing continuation of the procedure is made, the control unit 60 returns to step S8 and starts the puncture operation. In step S7, in a case where an instruction to end the automatic puncture by the blood vessel puncture system 10 is input, the control unit 60 causes the display unit 70 to display that the automatic puncture is terminated halfway, and terminates the automatic puncture.

In a case where it is determined in step S5 that the inner diameter of the blood vessel exceeds the threshold (or is equal to or greater than the threshold) or in a case where an input for instructing continuation of the automatic puncture is made in step S7, the control unit 60 determines that vasospasm does not occur or there is no problem even if vasospasm occurs, and starts the puncture operation as illustrated in FIG. 7. That is, the control unit 60 controls the first linear movement portion 42 and the second linear movement portion 48 to start integral movement of the needle 31 and the outer tube 33 toward the target puncture arrival position A (step S8).

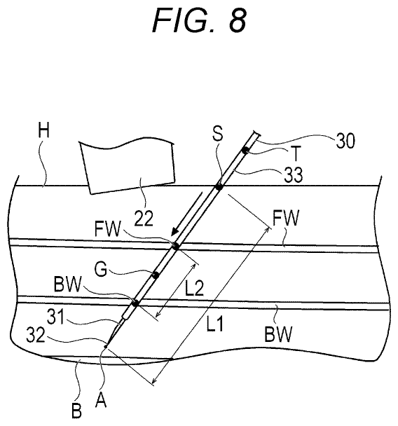

The control unit 60 determines whether the blood vessel inner diameter calculated from the image information acquired from the imaging unit 22 is equal to or less than a threshold (or less than the threshold) (step S9). In a case where it is determined in step S9 that the inner diameter of the blood vessel exceeds the threshold (or is equal to or greater than the threshold), the control unit 60 determines whether the needle 31 reaches the target puncture arrival position A while continuing the movement of the needle 31 and the outer tube 33 by determining that the vasospasm does not occur (step S10). In a case where it is determined in step S10 that the needle 31 has reached the target puncture arrival position A as illustrated in FIG. 8, the control unit 60 stops the drive of the first linear movement portion 42 and the second linear movement portion 48, and stops the puncture (step S11). In this state, the distal end of the needle 31 and the distal end of the outer tube 33 penetrate the back wall BW of the blood vessel. Subsequently, the control unit 60 drives the first linear movement portion 42 in a state where the second linear movement portion 48 is stopped, retracts the needle 31 in the opposite direction of the puncture direction while leaving the outer tube 33, and pulls out the outer tube 33 (step S12). At this time, since the distal end of the outer tube 33 penetrates the back wall BW, it is possible to suppress the occurrence of blood backflow through the lumen of the outer tube 33. As a result, the automatic puncture by the blood vessel puncture system 10 is completed.

Note that the control unit 60 may display that the puncture has been completed on the display unit 70 after step S11. In this case, the operation of removing the needle 31 from the outer tube 33 may be automatically performed under the control of the control unit 60, or may be manually performed by the operator.

After removing the needle 31 with the outer tube 33 being left, the operator inserts a guide wire from a proximal end opening of the outer tube hub 35 by a defined length. Subsequently, the operator removes the outer tube 33 with the guide wire being left, whereby a procedure for securing an access route to the blood vessel is completed.

In a case where it is determined in step S10 that the needle 31 has not reached the target puncture arrival position A, the control unit 60 continues driving of the first linear movement portion 42 and the second linear movement portion 48, and returns to step S9.

In a case where it is determined in step S9 that the inner diameter of the blood vessel is equal to or less than the threshold, the control unit 60 determines that there is a possibility that vasospasm has occurred, temporarily stops the movement of the needle 31 and the outer tube 33 (step S13), and displays a warning on the display unit 70 (step S14). Next, the control unit 60 waits for an input to the control unit 60 by the operator who has seen the warning (step S15). The operator checks the situation by viewing the warning and determines whether to continue the puncture as it is, continue dosing to eliminate vasospasm, or not continue the puncture. In step S15, in a case where an input for instructing continuation of puncture is made, the control unit 60 returns to step S8 and starts the puncture operation. In a case where an input for instructing not to continue the puncture is made in step S15, the control unit 60 causes the display unit 70 to display that the automatic puncture by the blood vessel puncture system 10 is terminated in the middle, and terminates the automatic puncture.

As described above, the blood vessel puncture apparatus 11 according to the first embodiment is a blood vessel puncture apparatus 11 connectable to the measurement unit (imaging unit 22) that measures the blood vessel diameter (for example, the inner diameter of the blood vessel) and the drive unit 40 that moves the needle 31 that performs puncture, and includes the control unit 60 that receives information (cross-sectional image) of the measurement result from the measurement unit and controls the operation of the drive unit 40. The control unit 60 determines whether the blood vessel contracts from the measurement result acquired from the measurement unit, and controls the drive unit 40 to start, continue, or stop the movement of the needle 31 in a case where it is determined that the blood vessel contracts. As a result, the blood vessel puncture apparatus 11 can detect vasospasm by detecting contraction of the blood vessel by the measurement unit (imaging unit 22) and start, continue, or stop puncture.

Furthermore, the blood vessel puncture apparatus 11 according to the first embodiment is a blood vessel puncture apparatus 11 connectable to the measurement unit (imaging unit 22) that measures the blood vessel diameter (for example, the inner diameter of the blood vessel), the drive unit 40 that moves the needle 31 that performs puncture, and an information transmission unit (display unit 70) that transmits information, and includes the control unit 60 that receives information of a measurement result (cross-sectional image) from the measurement unit and controls the operation of the drive unit 40. The control unit 60 determines whether the blood vessel contracts from the measurement result acquired from the measurement unit, and causes the information transmission unit (display unit 70) to transmit information indicating a warning in a case where it is determined that the blood vessel contracts. As a result, the blood vessel puncture apparatus 11 can detect vasospasm by detecting contraction of the blood vessel by the measurement unit and transmit a warning.

The measurement unit is the imaging unit 22 that comes into contact with the skin surface to acquire a cross-sectional image of the human body, and the control unit 60 calculates a blood vessel diameter from the cross-sectional image acquired from the imaging unit 22, and determines that the blood vessel contracts and vasospasm occurs in a case where the blood vessel diameter is equal to or less than a threshold. As a result, the blood vessel puncture apparatus 11 can effectively detect the vasospasm from the cross-sectional image acquired from the imaging unit 22.

Before starting the puncture operation by the drive unit 40, the control unit 60 calculates a threshold by multiplying the blood vessel diameter specified from the cross-sectional image acquired from the imaging unit 22 by a predetermined ratio. As a result, since the blood vessel puncture apparatus 11 calculates the threshold from the actual blood vessel diameter before puncture, the vasospasm can be effectively detected by appropriately setting the ratio.

The control unit 60 determines whether the blood vessel has contracted before the puncture operation by the drive unit 40 is started or during the puncture operation. As a result, the blood vessel puncture apparatus 11 can quickly detect the vasospasm even before or during the puncture of the blood vessel, and can safely cope with the vasospasm after the detection with time.

Furthermore, the method for controlling the blood vessel puncture system 10 according to the first embodiment is a method for controlling the blood vessel puncture system 10 including the measurement unit (imaging unit 22) that measures the blood vessel diameter (for example, the inner diameter of the blood vessel), the drive unit 40 that moves the needle 31 that performs puncture, and the blood vessel puncture apparatus 11 including the control unit 60 that receives information of the measurement result (cross-sectional image) from the measurement unit and controls the operation of the drive unit 40. The method includes step S9 of determining whether the blood vessel contracts from the measurement result acquired from the measurement unit, and step S13 of controlling the drive unit 40 to start, continue, or stop the movement of the needle 31 in a case where it is determined that the blood vessel contracts. As a result, the method for controlling the blood vessel puncture system 10 can detect vasospasm by detecting the contraction of the blood vessel by the measurement unit and start, continue, or stop the puncture.

Furthermore, the method for controlling the blood vessel puncture system 10 according to the first embodiment is a method for controlling the blood vessel puncture system 10 including the measurement unit (imaging unit 22) that measures the blood vessel diameter (for example, the inner diameter of the blood vessel), the drive unit 40 that moves the needle 31 that performs puncture, the information transmission unit (display unit 70) that transmits information, and the blood vessel puncture apparatus 11 including the control unit 60 that receives information of the measurement result (cross-sectional image) from the measurement unit and controls the operation of the drive unit 40. The method includes step S9 of determining whether the blood vessel contracts from the measurement result acquired from the measurement unit, and step S14 of causing the information transmission unit to transmit information indicating a warning in a case where it is determined that the blood vessel contracts. As a result, the method for controlling the blood vessel puncture system 10 can detect vasospasm by detecting contraction of the blood vessel by the measurement unit and transmit a warning.

### Second Embodiment

A blood vessel puncture system 10 according to a second embodiment of the present invention is different from that of the first embodiment in content of control in the control unit 60. In the second embodiment, the control unit 60 specifies the inner diameter of the blood vessel using the measurement result of the force sensor 80 (measurement unit).

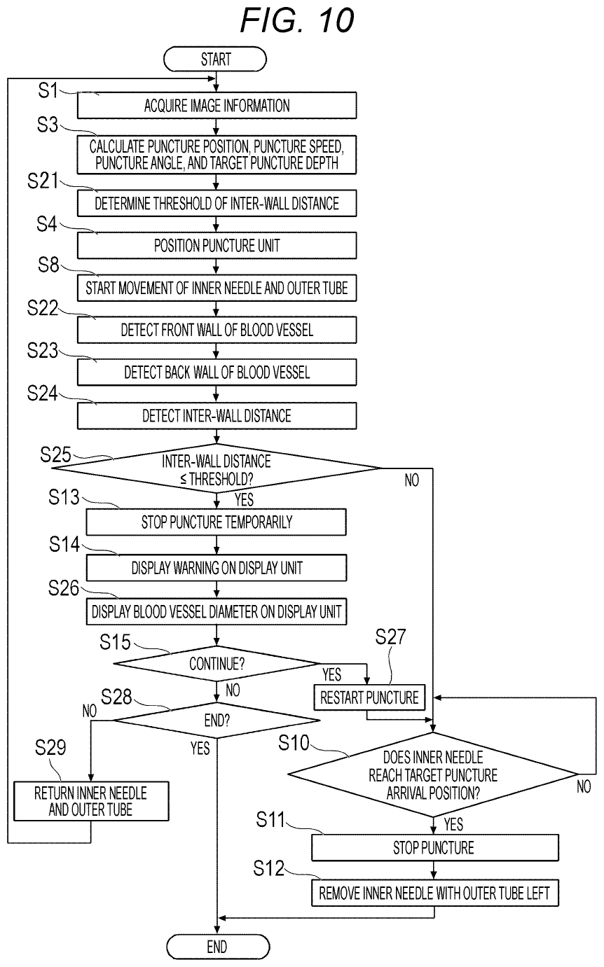

Next, a method for puncturing a blood vessel using the blood vessel puncture system 10 according to the second embodiment will be described with reference to a flowchart of the control unit 60 illustrated in FIG. 10. As illustrated in FIGS. 1 and 2, the blood vessel puncture system 10 is used in contact with the skin surface. Note that processes (steps) similar to those in the first embodiment are denoted by the same reference numerals, and description of the processes (steps) similar to those in the first embodiment will be omitted or simplified.

The control unit 60 acquires image information from the imaging unit 22 via the receiver 24 (step S1). The control unit 60 forms a cross-sectional image from the image information, and performs image analysis on the cross-sectional image to specify the position of the blood vessel in the image, and specifies the inner diameter of the blood vessel to determine a threshold (step S2). Next, as illustrated in FIGS. 5 and 6, the control unit 60 calculates a puncture position S, a puncture speed, a puncture angle θ, and a target puncture depth L1 of the skin surface from the position information of the blood vessel (step S3). Next, the control unit 60 specifies an inter-wall distance L2 from the front wall FW through which the needle 31 passes to the back wall BW from the cross-sectional image, the puncture position S, the puncture angle θ, and the like, and determines a threshold thereof (step S21).

The threshold of the inter-wall distance L2 is a value used to determine whether vasospasm has occurred. The threshold is a value obtained by multiplying the inter-wall distance L2 specified from the cross-sectional image by a ratio. Note that the threshold of the inter-wall distance L2 may be calculated from the outer diameter of the device to be inserted into the blood vessel after puncture, or may be a specific value determined by the operator.

Next, the control unit 60 controls and drives at least one of the first linear movement portion 42, the second linear movement portion 48, the third linear movement portion 45, the inclination portion 43, or the rotation portion 46. As a result, the puncture unit 30 is positioned at the desired position (coordinates) with the desired posture (angle) (step S4).

Next, the control unit 60 starts the puncture operation. That is, the control unit 60 controls the first linear movement portion 42 and the second linear movement portion 48 to start integral movement of the needle 31 and the outer tube 33 toward the target puncture arrival position A as illustrated in FIG. 7 (step S8).

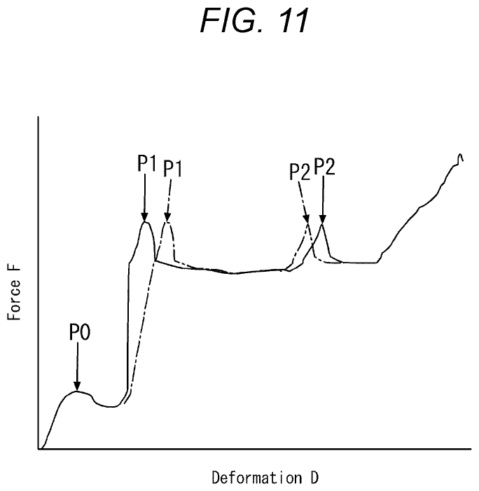

Next, the control unit 60 detects signals of the first linear movement portion 42 and the force sensor 80 (measurement unit) during the puncture operation, and monitors a puncture depth D (distance from the puncture position S to the distal end of the needle 31 during puncture) and the force F received by the needle 31. As illustrated in the graph of FIG. 11, the force F received by the needle 31 detected by the force sensor 80 rises when the needle tip 32 penetrates the skin and then falls to indicate a peak P0. Subsequently, the force F received by the needle 31 detected by the force sensor 80 rises when penetrating the front wall FW and then falls to indicate a peak P1. Subsequently, as illustrated in FIGS. 8 and 11, the force F received by the needle 31 detected by the force sensor 80 rises when the needle 31 penetrates the back wall BW and then falls to indicate a peak P2. Therefore, the control unit 60 can specify that the needle 31 has penetrated the skin, the front wall FW, and the back wall BW by monitoring the signal detected by the force sensor 80. The value of the peak P2 may be higher or lower than the value of the peak P1. The control unit 60 can specify a peak by determining whether the detected force F exceeds a predetermined value (absolute value), whether a change amount (difference value) of the detected force F exceeds a predetermined value, whether a gradient of the detected force F exceeds a predetermined value, or the like. Accordingly, the control unit 60 detects the front wall FW (step S22), and subsequently detects the back wall BW (step S23).

Next, the control unit 60 calculates the movement distance of the needle 31 from the time point the front wall FW is detected to the time point the back wall BW is detected as an inter-wall distance L2 (step S24), and determines whether the inter-wall distance L2 is equal to or less than a threshold (or less than the threshold) (step S25). When the vasospasm occurs, the inner diameter of the blood vessel decreases, and thus, the change in the puncture depth D from the peak P1 to the peak P2, that is, the movement distance of the needle 31 decreases, as indicated by an alternate long and short dash line in FIG. 11. Therefore, in a case where the inter-wall distance L2 decreases, there is a possibility that vasospasm has occurred.

In a case where it is determined in step S25 that the inter-wall distance L2 exceeds the threshold (or is equal to or greater than the threshold), the control unit 60 determines whether the needle 31 reaches the target puncture arrival position A while continuing the movement of the needle 31 and the outer tube 33 by determining that the vasospasm does not occur (step S10). The control unit 60 repeats step S10 while continuing the movement of the needle 31 and the outer tube 33 until it is determined in step S10 that the needle 31 has not reached the target puncture arrival position A. In a case where it is determined in step S10 that the needle 31 has reached the target puncture arrival position A, the control unit 60 stops the drive of the first linear movement portion 42 and the second linear movement portion 48, and stops the puncture (step S11). In this state, the distal end of the needle 31 and the distal end of the outer tube 33 penetrate the back wall BW of the blood vessel. Subsequently, the control unit 60 drives the first linear movement portion 42 in a state where the second linear movement portion 48 is stopped, retracts the needle 31 in the opposite direction of the puncture direction while leaving the outer tube 33, and pulls out the outer tube 33 (step S12). At this time, since the distal end of the outer tube 33 penetrates the back wall BW, it is possible to suppress the occurrence of blood backflow through the lumen of the outer tube 33. As a result, the automatic puncture by the blood vessel puncture system 10 is completed.

In a case where it is determined in step S25 that the inter-wall distance L2 is equal to or less than the threshold (or less than the threshold), the control unit 60 determines that there is a possibility that vasospasm has occurred, temporarily stops the movement of the needle 31 and the outer tube 33 (step S13), and displays a warning on the display unit 70 (step S14). Furthermore, the control unit 60 calculates the inner diameter of the blood vessel from the inter-wall distance L2 and displays the inner diameter on the display unit 70 (step S26). Next, the control unit 60 waits for an input to the control unit 60 by the operator who has seen the warning and the inner diameter (step S15). The operator checks the situation by viewing the warning and the inner diameter, and determines whether to continue puncturing. In a case where an input for instructing continuation of the procedure has been made in step S15, the control unit 60 restarts the driving of the first linear movement portion 42 and the second linear movement portion 48, restarts the puncture (step S27), and proceeds to step S10 described above for determining whether the needle 31 has reached the target puncture arrival position A.

In a case where an input for instructing not to continue puncturing has been made in step S15, the control unit 60 causes the display unit 70 to display that puncturing is not to be continued. Next, the control unit 60 causes the display unit 70 to request the operator to determine whether to end the puncture by the blood vessel puncture system 10, and waits for the operator to input to the control unit 60 (step S28). The operator checks the situation by viewing the display unit 70 and the like, and determines whether to end the automatic puncture by the blood vessel puncture system 10. In step S28, in a case where an instruction to end the automatic puncture by the blood vessel puncture system 10 is input, the control unit 60 ends the automatic puncture by the blood vessel puncture system 10.

In step S28, in a case where an instruction not to end the automatic puncture by the blood vessel puncture system 10 is input, the control unit 60 drives the first linear movement portion 42 and the second linear movement portion 48 to return the needle 31 and the outer tube 33 to the original positions before puncture (step S29). Next, the control unit 60 returns to step S1 and can perform the automatic puncture again.

As described above, in the second embodiment, the measurement unit is the force sensor 80 that detects the force acting on the needle, and the control unit 60 specifies the first peak P1 when the needle 31 punctures the front wall FW of the blood vessel and the second peak P2 when puncturing the back wall BW of the blood vessel after the first peak P1 from the detection result of the force sensor 80, calculates the inter-wall distance L2 that is the movement amount of the needle 31 from the first peak P1 to the second peak P2, and determines that the blood vessel is contracted and the vasospasm occurs in a case where the inter-wall distance L2 is equal to or less than the threshold. As a result, the blood vessel puncture apparatus 11 can effectively detect the vasospasm from the detection result of the force acquired from the force sensor 80.

Before starting the puncturing operation by the drive unit 40, the control unit 60 calculates the threshold by multiplying the distance between the front wall FW and the back wall BW of the blood vessel specified from the cross-sectional image acquired from the imaging unit 22 that comes into contact with the skin surface and acquires the cross-sectional image of the human body by a predetermined ratio. Accordingly, since the blood vessel puncture apparatus 11 calculates the threshold from the distance between the front wall FW and the back wall BW of the actual blood vessel before puncture, the vasospasm can be effectively detected by appropriately setting the ratio.

Note that, the present disclosure is not limited to the embodiment described above, and various modifications can be made by those skilled in the art within the technical idea of the present disclosure. For example, the drive unit 40 has five movable portions (the first linear movement portion 42, the second linear movement portion 48, the third linear movement portion 45, the rotation portion 46, and the inclination portion 43), but the number of movable portions may be six or more or four or less. In addition, the drive unit 40 may be a robot arm. Also, the needle 31 and the outer tube 33 may be configured to move integrally by the same drive source rather than being driven separately.

The detailed description above describes embodiments of a blood vessel puncture apparatus capable of automatically puncturing a blood vessel and a method for controlling a blood vessel puncture system. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A blood vessel puncture apparatus connectable to a measurement unit configured to measure a blood vessel diameter and a drive unit configured to move a needle for puncturing, the blood vessel puncture apparatus comprising:
a control unit configured to:
receive information of a measurement result from the measurement unit and control an operation of the drive unit; and
determine whether the blood vessel contracts from a measurement result acquired from the measurement unit, and control the drive unit to start, continue, or stop movement of the needle in a case where it is determined that the blood vessel contracts.

2. The blood vessel puncture apparatus according to claim 1, wherein
the measurement unit is an imaging unit that is configured to come into contact with a skin surface to acquire a cross-sectional image of a human body; and
the control unit is configured to:
calculate a blood vessel diameter from a cross-sectional image acquired from the imaging unit; and
determine, in a case where the blood vessel diameter is equal to or less than a threshold, that the blood vessel contracts and vasospasm occurs.

3. The blood vessel puncture apparatus according to claim 1, wherein
the measurement unit is a force sensor configured to detect a force acting on the needle; and
the control unit is configured to:
specify a first peak when the needle punctures a front wall of a blood vessel and a second peak when the needle punctures a back wall of the blood vessel after the first peak from a detection result of the force sensor;
calculate an inter-wall distance that is a movement amount of the needle between the first peak and the second peak; and
determine, in a case where the inter-wall distance is equal to or less than a threshold, that the blood vessel contracts and vasospasm occurs.

4. The blood vessel puncture apparatus according to claim 2, wherein the control unit is configured to calculate the threshold by multiplying a blood vessel diameter specified from a cross-sectional image acquired from the imaging unit by a predetermined ratio before starting a puncture operation by the drive unit.

5. The blood vessel puncture apparatus according to claim 3, wherein before starting a puncture operation by the drive unit, the control unit is configured to calculate the threshold by multiplying a distance between a front wall and a back wall of a blood vessel specified from a cross-sectional image acquired from an imaging unit that is configured to come into contact with a skin surface and acquires a cross-sectional image of a human body by a predetermined ratio.

6. The blood vessel puncture apparatus according to claim 1, wherein the control unit is configured to determine whether the blood vessel contracts before starting a puncture operation by the drive unit or during the puncture operation.

7. A blood vessel puncture apparatus connectable to a measurement unit configured to measure a blood vessel diameter, a drive unit configured to move a needle for puncturing, and an information transmission unit configured to transmit information, the blood vessel puncture apparatus comprising:
a control unit configured to:
receive information of a measurement result from the measurement unit and control an operation of the drive unit; and
determine whether a blood vessel contracts from a measurement result acquired from the measurement unit, and cause the information transmission unit to transmit information indicating a warning in a case where it is determined that the blood vessel contracts.

8. The blood vessel puncture apparatus according to claim 7, wherein
the measurement unit is an imaging unit configured to come into contact with a skin surface to acquire a cross-sectional image of a human body; and
the control unit is configured to:
calculate a blood vessel diameter from a cross-sectional image acquired from the imaging unit; and
determine, in a case where the blood vessel diameter is equal to or less than a threshold, that the blood vessel contracts and vasospasm occurs.

9. The blood vessel puncture apparatus according to claim 7, wherein
the measurement unit is a force sensor configured to detect a force acting on the needle; and
the control unit is configured to:
specify a first peak when the needle punctures a front wall of a blood vessel and a second peak when the needle punctures a back wall of the blood vessel after the first peak from a detection result of the force sensor;
calculate an inter-wall distance that is a movement amount of the needle between the first peak and the second peak; and
determine, in a case where the inter-wall distance is equal to or less than a threshold, that the blood vessel contracts and vasospasm occurs.

10. The blood vessel puncture apparatus according to claim 8, wherein the control unit is configured to calculate the threshold by multiplying a blood vessel diameter specified from a cross-sectional image acquired from the imaging unit by a predetermined ratio before starting a puncture operation by the drive unit.

11. The blood vessel puncture apparatus according to claim 9, wherein before starting a puncture operation by the drive unit, the control unit is configured to calculate the threshold by multiplying a distance between a front wall and a back wall of a blood vessel specified from a cross-sectional image acquired from an imaging unit configured to come into contact with a skin surface and acquires a cross-sectional image of a human body by a predetermined ratio.

12. The blood vessel puncture apparatus according to claim 7, wherein the control unit is configured to determine whether the blood vessel contracts before starting a puncture operation by the drive unit or during the puncture operation.

13. A method for controlling a blood vessel puncture system including a measurement unit that measures a blood vessel diameter, a drive unit that moves a needle for puncturing, an information transmission unit that transmits information, and a blood vessel puncture apparatus including a control unit that receives information of a measurement result from the measurement unit and controls an operation of the drive unit, the method comprising:
determining whether a blood vessel contracts from a measurement result acquired from the measurement unit; and
causing the information transmission unit to transmit information indicating a warning in a case where it is determined that the blood vessel contracts.

14. The method according to claim 13, further comprising:
controlling, by the control unit, the drive unit to start, continue, or stop movement of the needle in a case where it is determined that the blood vessel contracts.

15. The method according to claim 13, wherein the measurement unit is an imaging unit that comes into contact with a skin surface to acquire a cross-sectional image of a human body.

16. The method according to claim 15, further comprising:
calculating, by the control unit, a blood vessel diameter from a cross-sectional image acquired from the imaging unit; and
determining, by the control unit, in a case where the blood vessel diameter is equal to or less than a threshold, that the blood vessel contracts and vasospasm occurs.

17. The method according to claim 16, further comprising:
calculating the threshold by multiplying a blood vessel diameter specified from a cross-sectional image acquired from the imaging unit by a predetermined ratio before starting a puncture operation by the drive unit.

18. The method according to claim 13, wherein the measurement unit is a force sensor that detects a force acting on the needle, and the method further comprises:
specifying, by the control unit, a first peak when the needle punctures a front wall of a blood vessel and a second peak when the needle punctures a back wall of the blood vessel after the first peak from a detection result of the force sensor;
calculating, by the control unit, an inter-wall distance that is a movement amount of the needle between the first peak and the second peak; and
determining, by the control unit, in a case where the inter-wall distance is equal to or less than a threshold, that the blood vessel contracts and vasospasm occurs.

19. The method according to claim 17, wherein before starting a puncture operation by the drive unit, the method further comprises:
calculating, by the control unit, the threshold by multiplying a distance between a front wall and a back wall of a blood vessel specified from a cross-sectional image acquired from an imaging unit that comes into contact with a skin surface and acquires a cross-sectional image of a human body by a predetermined ratio.

20. The method according to claim 13, further comprising:
determining, by the control unit, whether the blood vessel contracts before starting a puncture operation by the drive unit or during the puncture operation.
